(19) United States
(12) Patent Application Publication
(10) Pub. No.: US 2002/0170771 A1
(43) Pub. Date: Nov. 21, 2002

(54) PROTECTIVE COVER FOR STETHOSCOPE, AND DISPENSING ASSEMBLY COMPRISING SAME

(75) Inventors: Michael W. Milam, Sarasota, FL (US); Tilak M. Shah, Cary, NC (US); Dezso K. Levius, Cary, NC (US)

Correspondence Address: Steven J. Hultquist, Intellectual Property/Technology Law, P.O. Box 14329, Research Triangle Park, NC, 27709, US

(73) Assignee: Infection Control Solutions, Inc., Sarasota, FL (US)

(21) Appl. No.: 09/855,364

(22) Filed: May 15, 2001

(51) Int. Cl. A61B 7/02

(52) U.S. Cl. 181/131; 181/130

(57) ABSTRACT

A protective cover for an article such as a stethoscope, comprising a bag member of elongate form, closed at a first end and fully or partially open at a second, opposite end to form an enclosed volume for retention of the head and elongate connector body of a stethoscope. The two longitudinal sides of such bag member can be seamless; alternatively, such longitudinal sides can comprise two seam lines. The bag member is longitudinally, medially severable at an end portion that include the second, open end, to yield two free end segments for covering respective ear tubes of the stethoscope. The protective cover may further include retention means for positionally retaining the free end segments of the bag member in protective covering relationship to the ear tubes of the stethoscope, e.g., adhesive and/or compressive clips, or a transverse heat seal at marginal extremities of the free end segments.

### BACKGROUND OF THE INVENTION

1. Field of the Invention

The present invention relates to a protective cover useful for preventing stethoscopic contamination and transmission of infectious organisms and other biohazards, as well as to an assembly for dispensing same.

2. Description of the Related Art

Use of stethoscopes by health care professionals during patient examinations has become a known cause of cross-contamination and biohazards transmission in today's hospitals. The stethoscope, typically a personal device carried by a health care professional, is used to examine multiple patients, and during each examination process, the stethoscope directly contacts body parts of each patient, and it acts therefore as a fomite from patient to patient, significantly increasing cross-contamination and transmission of infectious organisms.

Single-use stethoscopes have been implemented as a solution to eliminate breaks in barrier protection. However, these stethoscopes are often left in the room of patients carrying resistant organisms and quickly become contaminated. More importantly, the single-use stethoscope is hardly ever used in place of the health care professional's personal stethoscope. As a result, the single-use stethoscope has not been effective in infection control isolation and control.

Cleaning of stethoscopes after each use is another approach to avoid cross-contamination and biohazards transmission caused by stethoscopes. However, such cleaning process is ineffective in eliminating certain resistant infectious organisms, and it is also difficult for the health care professionals to carry out the cleaning process each time with consistency.

A number of other approaches to control the problem of microorganism transmission via the stethoscope have been developed. U.S. Pat. Nos. 4,867,265, 5,486,659, 5,564,431, 5,747,751, and 5,813,992 disclose a cover for isolating the stethoscope and preventing it from directly contacting skin of the patients. However, such cover only extends over the head of the stethoscope, and the rest of the stethoscope remains exposed and susceptible to contamination.

U.S. Pat. No. 5,623,131 discloses a two-piece stethoscope cover that has a first piece that covers the head and a second piece that covers the elongate connector tube that connects the head to the bifurcated ear tubes. However, the ear tubes remain exposed, and there is also an exposed region between the two pieces in some embodiments. Moreover, it is time-consuming and cumbersome for the health care professionals to have to place both pieces onto the stethoscope properly before starting the examination, especially in case of emergency situations.

U.S. Pat. No. 5,592,946 discloses a stethoscope cover that leaves both the ear tubes and the stethoscope head exposed. U.S. Pat. No. 5,466,898 discloses a stethoscope isolation system that, although completely covering the stethoscope head, leaves most of the ear tubes exposed. U.S. Design Pat. No. 376,043 shows a stethoscope cover that does extend up the length of the ear tubes. This cover does not protect the side of the head of the stethoscope that contacts the patient.

U.S. Pat. No. 6,186,957 entitled “Stethoscope Cover” issued Feb. 13, 2001 in the name of Michael W. Milam discloses a stethoscope cover that comprising a front panel and a back panel, which collectively form a pouch with a closed distal end and an open proximal end, configured to receive the head and the connector portion of the stethoscope. Such stethoscope cover also comprises a first flap and a second flap, having a “Y” shape accommodated for covering the two bifurcated ear tubes. However, mass manufacturing of such stethoscope covers of irregular shape is difficult. Moreover, it is hard to pack multiple such stethoscope covers in a manner that enables easy and quick access to a supply of multiple covers of such type.

It therefore would be a significant advance in the art to provide a stethoscope cover that is easy to use, easy to manufacture, and readily dispensable, without sacrificing its effectiveness in preventing cross-contamination and transmission of infectious organisms during use of the stethoscope.

### SUMMARY OF THE INVENTION

The present invention relates to a protective cover for a stethoscope or like article.

The stethoscope to which the stethoscope cover of the invention is advantageously applied may be of any suitable type, and may be of conventional form including a stethoscope head, an elongate connector body portion, and first and second ear tubes.

In one embodiment, the protective cover comprises an elongate bag member, which may be of any suitable shape, e.g., rectangular, square, trapezium, truncated ellipse with flat ends, etc. The bag has a first and a second end and is closed at the first end thereof, while open at the second, opposite end thereof. Such bag member defines an enclosed interior volume with an opening at the second, opposite end, into which the head and the elongate connector body portion of a stethoscope or like article can be reposed. The bag member is also longitudinally, medially severable along an end portion thereof that includes the second, opposite open end. By applying tensional force at opposing margins of the end portion of the bag member, the bag member is partially severed to yield two free end segments at the end portion, and such free end segments are readily positionable to cover the first and second ear tubes of a stethoscope. The bag member and its deployed free end segments therefore are adapted to protectively cover the whole stethoscope and effectively prevent contamination of such stethoscope from or to the body of the patient being examined.

The protective cover article may be formed of any material, preferably comprising a material that is acoustically transmitting and impermeable to bacteria, viruses and fluids. For example, the protective cover can be formed of a suitable thermoplastic polymeric material, such as polyurethane, polyethylene, polypropylene, polyvinyl, latex, silicone, etc. Various methods known in the field of the art can be employed to form closure at the first end of the bag member. Such methods include, but are not limited to, heat sealing, radio-frequency sealing, ultrasonic welding, and adhesive bonding.

The bag member may be scored along the end portion that includes the second, open end, so that by applying tensional force at opposing margin of such end portion, a health care professional can easily severe such end portion of the bag member longitudinally and medially, to yield the two free end segment for covering the bifurcated ear tubes of the stethoscope. More specifically, the bag member may comprise a central tear line along the end portion that includes the second, open end, so that the bag member is partially severable along such tear line.

In order to ensure attachment of the protective cover to the stethoscope during patient examination and prevent potential contamination caused by dropping of such cover, the protective cover can comprise means for removably securing the bag member to the stethoscope, so that the bag member is temporarily secured during patient examination and can be readily removed from the stethoscope after the examination is completed.

Particularly, it is preferred that the protective cover comprises means for associatively retaining the two free end segments of the bag member to respective ear tubes, so that the free end segments are in protective covering relationship with the ear tubes during the examination process. One preferred embodiment of such protective cover comprises transverse heat seals at extremities of the free end segments. Another preferred embodiment comprises compressive retention elements. Any suitable compressive retention elements can be use, which includes but is not limited to, compressive fasteners, snap buttons, and clips. Clips with adhesive applied thereto are preferred, e.g., an adhesive patch covered with a manually peelable strip that is readily removed to expose the adhesive surface. In such embodiments, the adhesive functions to adhesively secure the clips onto the bag member, so that the clips will not be scattered or present a trash or refuse problem after the bag member is removed from the stethoscope for disposal.

In another aspect, the present invention relates to a protectively covered stethoscope assembly, which comprises:

a protective cover as described hereinabove, and

a stethoscope having a stethoscope head, an elongate connector body portion, and first and second ear tubes,

wherein the stethoscope head and the elongate connector body portion are disposed in the interior volume of the bag member, and wherein the first and second ear tubes are disposed in respective free end segments of the bag member, so that the stethoscope is substantially shrouded by the bag member to prevent contamination by or transmission of biohazardous materials.

A further aspect of the present invention relates to a receptacle for dispensing multiple above-described protective covers for stethoscopes or like articles, comprising:

a roll member that comprises a continuous elongate strip of multiple protective covers as described hereinabove, which are in lay-flat film form and arranged in series-connected relationship, with a transverse tear line between every two adjacent protective covers, while such transverse tear line comprises a central slit; and

a box enclosure in which the roll member is disposed, having:

(a) a top hingedly attached thereto, which comprises a tongue member capable of protruding exteriorly of the box enclosure for engaging the central slit of the transverse tear line between every two adjacent protective covers;

(b) a slot opening in a side wall of the box enclosure, through which a leading end portion of the continuous elongate strip of multiple protective covers is drawable, in such manner that the central slits of successive tear lines of the continuous elongate strip are in sequential register with the exteriorly protruding tongue member, whereby application of tensional force longitudinally along said continuous elongate strip effects severing of at least one protective cover from a rearwardly next-succeeding protective cover in the continuous elongate strip.

The box enclosure of the receptacle can be formed of any material suitable for the practice of the present invention. Examples of useful materials for making the box enclosure include paperboard, metal or metal alloys, plastics, wood board, cardboard, stiffened/impregnated fabrics, etc.

In a specific embodiment of the present invention, the top of the box enclosure comprises a front flange. The tongue member is depending outwardly from the front flange and is also foldable against such front flange, so that when the front flange is “inserted into” the box enclosure, i.e., being interiorly positioned against a front wall of the box enclosure, the tongue member will “stick out”, i.e., protrude vertically above the top of the box enclosure, for engaging the central slit of the transverse tear line between every two adjacent protective covers.

The slot opening, from which the continuous elongate strip of protective covers is drawn, can be positioned either in a front wall or a rear wall of the box enclosure, as long as the central slits of successive tear lines of the continuous elongate strip are in sequential register with the exteriorly protruding tongue member.

In a preferred embodiment of the present invention, the box enclosure comprises a side compartment, which provides space for containing a supply of the compressive retention elements (i.e. compressive fasteners, snap buttons, clips, etc.). In such manner, the compressive retention elements are readily accessible to a health care professional for retaining the free end segments of the protective cover to respective ear tubes of the stethoscope. Such side compartment may have opening at the top of the box enclosure. Alternatively, it may have opening at a sidewall of the box enclosure.

Yet another aspect of the present invention relates to a method of protectively sheathing a stethoscope to prevent contamination by or transmission of biohazardous material in use of the stethoscope during a patient examination procedure, comprising protectively sheathing said stethoscope in a protective cover as described hereinabove, prior to the patient examination procedure.

Various other aspects, features and embodiments of the invention will be more fully apparent from the ensuing disclosure and claims.

### BRIEF DESCRIPTION OF THE DRAWINGS

FIG. 1 is a front view of a protective cover according to one embodiment of the present invention.

FIG. 2 is a front view of the protective cover in FIG. 1, after being partially severed along a central tear line to yield two free end segments.

FIG. 3 is a front view of the protective cover in FIG. 2, in protective covering relationship with a stethoscope reposed in such cover.

FIGS. 4-6 are cross-sectional views of various clip members useful in the practice of the present invention.

FIG. 7 is a perspective view of a box enclosure according to one embodiment of the present invention.

FIG. 8 is a cross-sectional view of the box enclosure in FIG. 7 from line I-I, having its top partially lifted to show details thereof.

FIG. 9 is an elevational view of a roll member comprising a continuous strip of multiple protective covers.

FIG. 10 is a perspective view of a receptacle comprising both a roll member and a box enclosure for dispensing protective covers of the present invention.

FIG. 11 is a front view of a protective cover according to another embodiment of the present invention.

FIG. 12 is a front view of the protective cover in FIG. 11, after being partially severed along a central tear line to yield two free end segments.

FIG. 13 is a front view of the protective cover in FIG. 12, in protective covering relationship with a stethoscope reposed in such cover.

FIG. 14 is a front view of a protective cover according to one embodiment of the present invention.

FIG. 15 is a front view of a protective cover according to another embodiment of the present invention.

### DETAILED DESCRIPTION OF THE INVENTION, AND PREFERRED EMBODIMENTS THEREOF

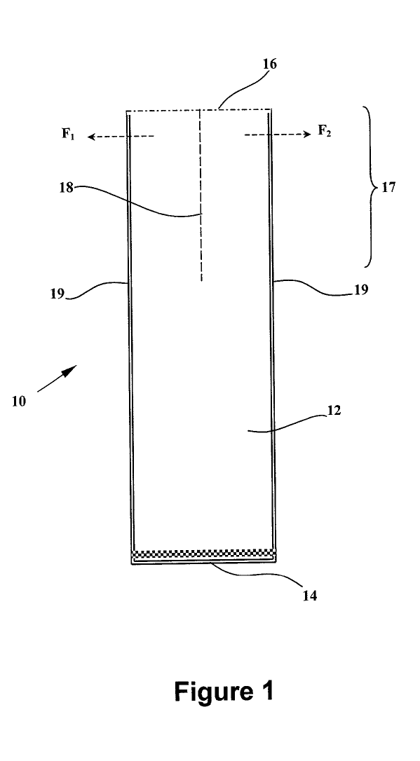

Referring to FIG. 1, a protective cover 10 comprises an elongate bag member 12. The bag member 12 is closed at a first end 14 and open at a second, opposite end 16. The first end 14 may be closed by any method or technique known in the art, such as heating sealing, RF sealing, ultrasonic welding, or adhesive bonding. Alternatively, such close end 14 may be formed by simply folding an elongate strip of polymeric film and sealing such film at its two longitudinal sides, so that the folding edge of such film naturally forms the close end 14.

The bag member 12 defines an enclosed interior volume with an opening at the second end 16. The two longitudinal sides 19 of the bag member 12 can be formed either with seam lines or without seam lines.

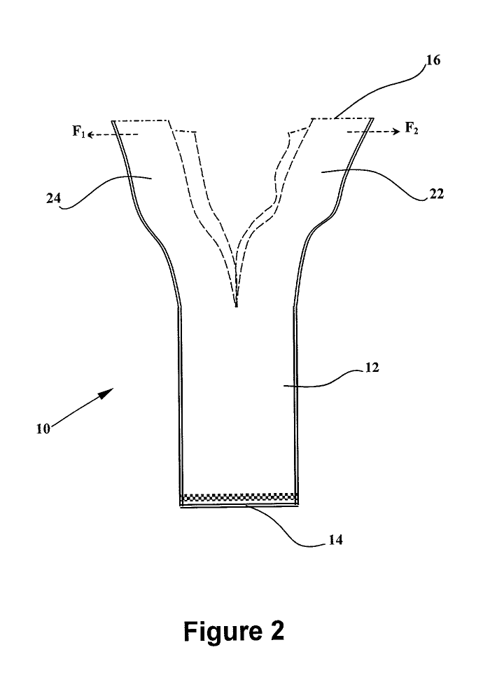

A central tear line 18 is scored on the bag member 12 along an end portion 17 that includes the second, open end 16, so when tensional forces F₁ and F₂ are applied at opposing margins of the end portion 17, the bag member can be longitudinally, medially severed along such end portion 17, to yield two free end segments 22 and 24, as shown in FIG. 2. These free end segments 22 and 24 accommodate the bifurcated shape of a stethoscope and can substantially cover the two ear tubes of the stethoscope.

Although FIGS. 1 and 2 show a bag member 12 of a perfect rectangular shape, the shape of such bag member is not thus limited. It can be any other suitable shape, such as square, trapezium, truncated ellipse with flat ends, etc. The open end 16 can be either longer than the close end 14, or shorter than the close end 14. The two longitudinal sides 19 of the bag member 12 can either be either straight, or curved, with concave or convex shape. Multiple variations in respect to the shape of the bag member 12 can be readily determined by one skilled in the art without undue experimentation and are within the scope and spirit of the present application.

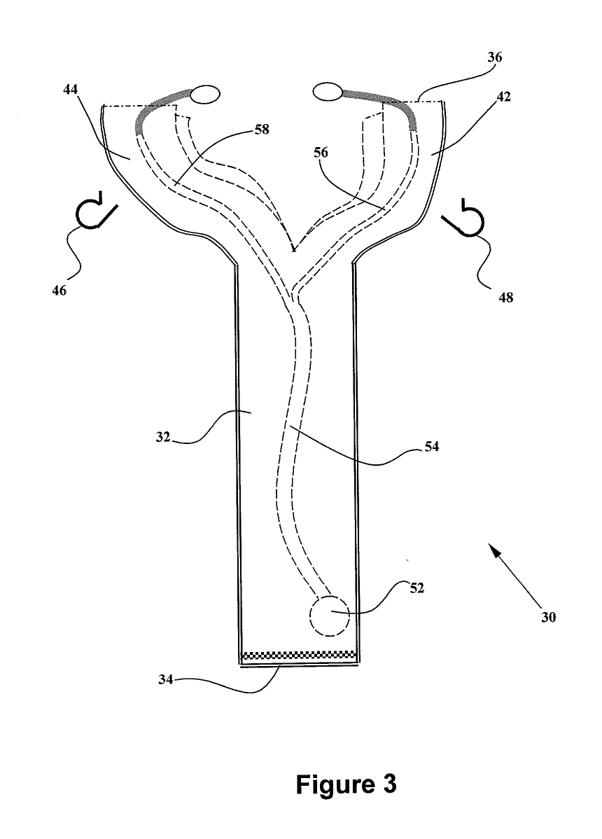

FIG. 3 shows a protectively covered stethoscope assembly 30, which comprises a protective cover 32 with a first, closed end 34 and a second, opposing open end 36, and a stethoscope with a stethoscope head 52, an elongate connector portion 54, and bifurcated ear tubes 56 and 58. The protective cover 32 has already been longitudinally, medially severed to generate two free end segments 42 and 44.

The stethoscope head 52 and the elongate connector body portion 54 of the stethoscope are disposed in the interior volume of the protective cover 32, while the two ear tubes 56 and 58 of the stethoscope are disposed in respective free end segments 42 and 44. In such manner, the stethoscope is protectively covered in all its parts and effectively isolated from bio-contaminants.

Two compressive retention elements 46 and 48 can be used to secure the two free end segments 42 and 44 to respective ear tubes 56 and 58 to prevent such free end segments from being detached from the ear tubes during patient examination.

Such compressive retention elements may be of any form and of any material. In one preferred embodiment of the present invention, such retention elements are clip members.

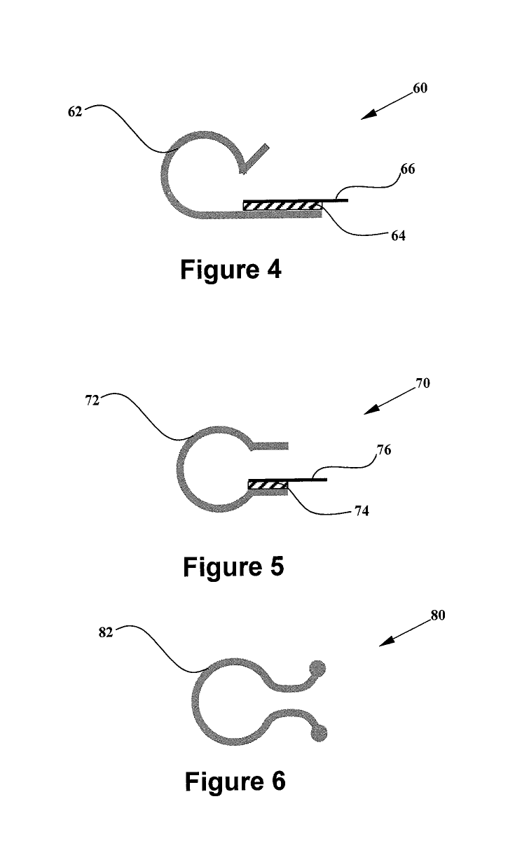

FIG. 4 shows a clip member 60 with a compressible clip body 62 that can be readily clipped onto an ear tube of a stethoscope. Such clip member 60 also comprises a layer of adhesive 64 covered by a removable paper top 66. During use of such clip member 60, the removable paper top 66 is peeled off to expose the adhesive layer 64 underneath. The adhesive layer 64 adhesively secures the clip member 60 to a protective cover, so after the protective cover is removed from the stethoscope upon completion of an examination and disposed, the clip member 60 remain attached to the protective cover, without being scattered in the disposal process, and presenting a trash or refuse problem. By such unitary structure including affixed clip elements, the cover article is readily and unitarily disposable.

FIG. 5 shows another clip member 70 with a compressible clip body 72, a layer of adhesive 74, and a removable paper top element 76.

Clip members without an adhesive layer may also be used for practicing of the present invention. For example, FIG. 6 shows a simple clip member 80 with just a clip body 82.

The protective covers of the present invention may be massively manufactured in a lay-flat film form. Multiple such protective covers are arranged in series-connected relationship, with the first, closed end of one protective cover immediately connects to the second, open end of the next, succeeding protective cover, in a “head-to-tail” manner, therefore forming a continuous elongate strip of protective covers. Such continuous elongate strip can then be rolled up to form a compact roll member that can be easily stored or transported. In order to facilitate easy dispensing of individual protective covers from such a roll member, transverse tear lines can be scored between every two adjacent protective covers, so that application of tensional force longitudinally along the continuous elongate strip can readily effect separation of a proactive cover from such strip. More preferably, the transverse tear lines comprise central slits, whose use will be discussed hereinafter in greater detail.

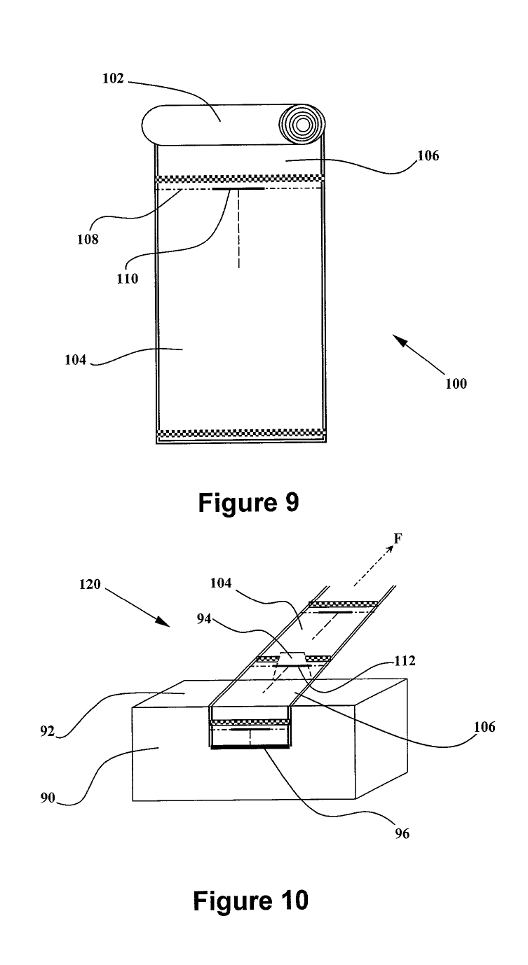

FIG. 9 shows a roll member 100 that comprises a continuous elongate strip 102 of multiple protective covers, which are in lay-flat film form. Every two adjacent protective covers 104 and 106 are arranged in a “head-to-tail” fashion, having a transverse tear line 108 therebetween. Such transverse tear line 108 preferably comprises a central slit 110.

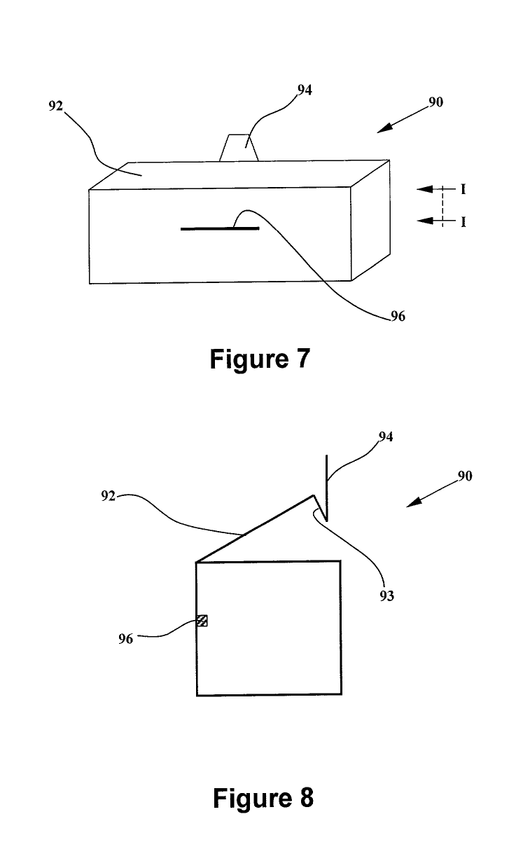

FIG. 7 shows a box enclosure 90, according to one embodiment of the present invention, for containing the roll member as described hereinabove. Such box enclosure comprises a top 92, which is hingedly attached thereto, see FIG. 8, which shows such top being hingedly lifted up. The top 92 comprises a tongue member 94 that protrudes exteriorly of the box enclosure 90. Such tongue member 94 is configured and dimensioned to engage the central slits 110 of the transverse tear lines 108 on the roll member 100 as shown in FIG. 9.

The box enclosure 90 also comprises a slot opening 96 in one sidewall. Through such slot opening 96, the continuous elongate strip 102 as shown in FIG. 9 can be drawn. The central slits of successive tear lines of the continuous elongate strip, being in sequential register with the exteriorly protruding tongue member 94 of the box enclosure, can therefore be fit onto the tongue member.

FIG. 10 shows a receptacle 120 that comprises both the roll member as in FIG. 9 (partially shown) and the box enclosure as in FIG. 7. The continuous elongate strip of the roll member is drawn through the slot opening 96 in a rear sidewall (or a front sidewall) of the box enclosure 90. The box enclosure 90 comprises a hingedly attached top 92 with a tongue member 94. The transverse tear line between two adjacent protective covers 104 and 106 of the continuous elongate strip comprises a central slit 112, which is in register with and has been fit onto the tongue member 94 of the box enclosure. By applying a tensional force F in longitudinal direction along the continuous elongate strip, protective cover 104 can be separated from the rearwardly next-succeeding protective cover 106.

The box enclosure of the present invention preferably comprises a front flange 93, as shown in FIG. 8. Such front flange 93 supports the tongue member 94, while the tongue member 94 is foldable against the front flange 93. Therefore, when the front flange 93 is “inserted into” the box enclosure and interiorly positioned against a front wall of the box enclosure, the tongue member 94 flips upwardly and protrudes vertically above the top 92 of the box enclosure.

The box enclosure may be alternatively constructed with an extendible tongue structure in other embodiments. For example, the tongue member may comprise a rigid element that is normally in a retracted position, flat against a panel (e.g., side, end, top) of the box enclosure, and mounted under retention elements allowing sliding movement of the tongue member to an extended, deployed position (protruding beyond a corresponding edge of the panel on which the tongue member is mounted). Other conformations of the tongue member are readily implemented, as will suggest themselves to those skilled in the art.

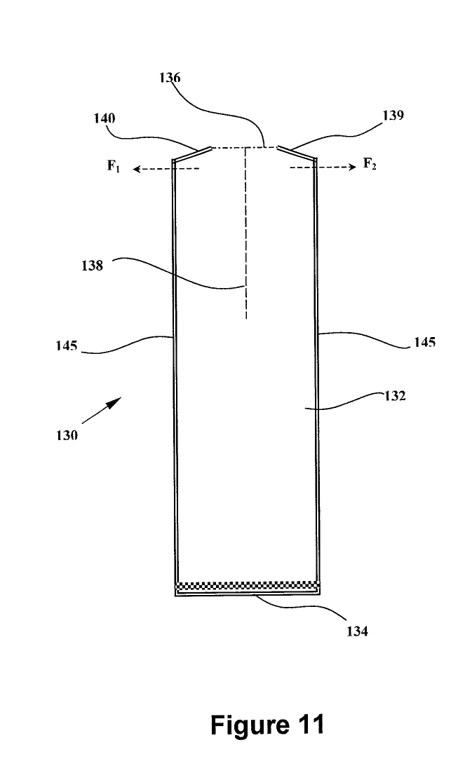

The protective cover of the present invention, in an alternative embodiment, may comprises transverse heat seals at extremities of the two free end segments, as shown in FIG. 11. The protective cover 130, as described above, comprises an elongate bag member 132 with a first, closed end 134, and a second, open end 136, and having two longitudinal sides 145 either with or without seam lines. A central tear line is provided for easy severance of such bag member longitudinally, medially into two free end segments 142 and 144. At the extremities of the two free end segments 142 and 144, two transverse heat seals 140 or 139 are provided, so that the free end segments 142 and 144 will automatically “hang on” to respectively ear tubes of the stethoscope, and no retention means is necessary for securing such free end segments thereto.

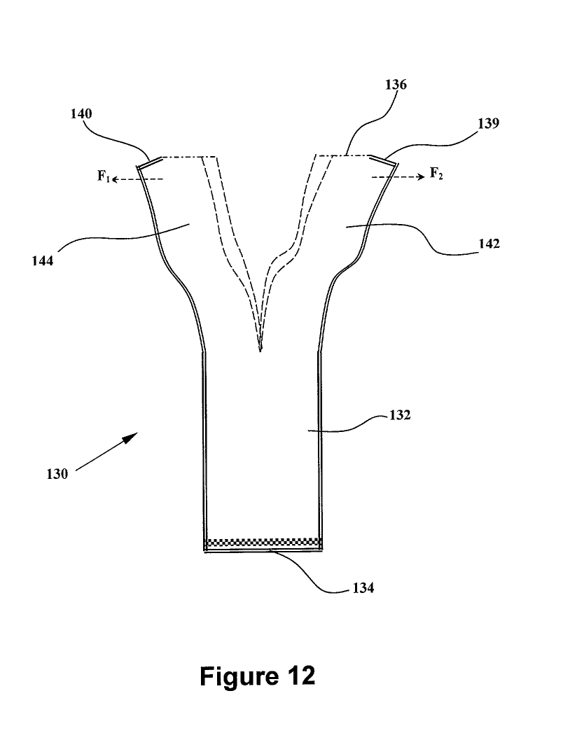

FIG. 12 shows the protective cover 130 of FIG. 11, being torn open by tensional forces F₁ and F₂ along the central tear line 138, so that such protective cover 130 is ready to be applied to a stethoscope for protective isolation of such stethoscope.

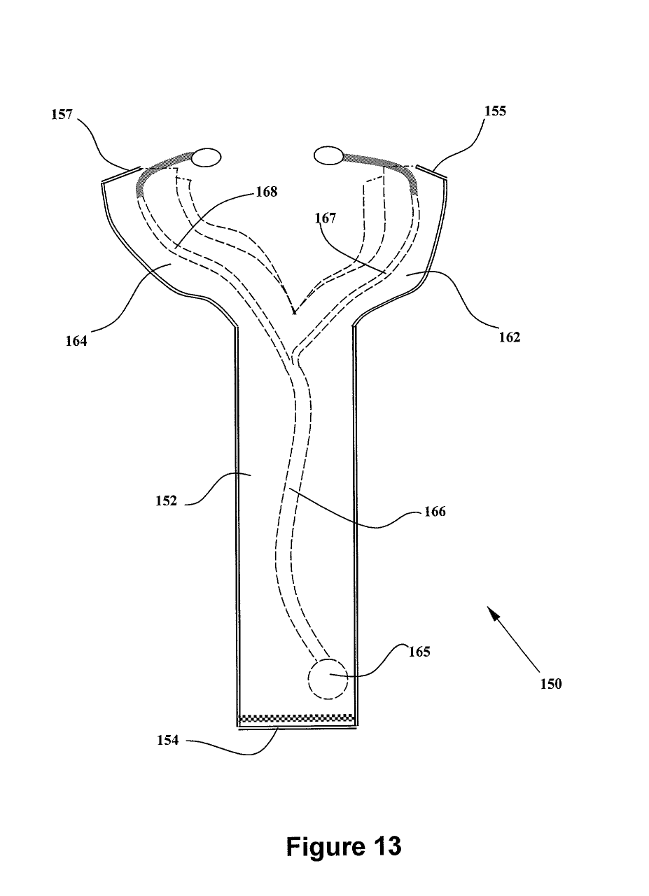

FIG. 13 shows a protectively covered stethoscope assembly 150, which comprises a protective cover 152 closed at one end 154. Such protective cover 152 defines an enclosed interior volume, within which reposed are a stethoscope that has a stethoscope head 165, an elongate connector portion 166, and bifurcated ear tubes 167 and 168. The protective cover 152 has already been longitudinally, medially severed to generate two free end segments 162 and 164, which can cover substantial portion or all of the bifurcated ear tubes 167 and 168.

The protective cover 152 comprises two transverse seals 155 and 157 at extremities of the two free end segments 162 and 164, respectively. Such transverse seals form “end hooks” that hook the two free end segments 162 and 164 to the ear tubes, so that such free end segment will not drop off during the patient examination process, and no additional retention elements are needed for retaining such free end segments 162 and 164 to the ear tubes 167 and 168.

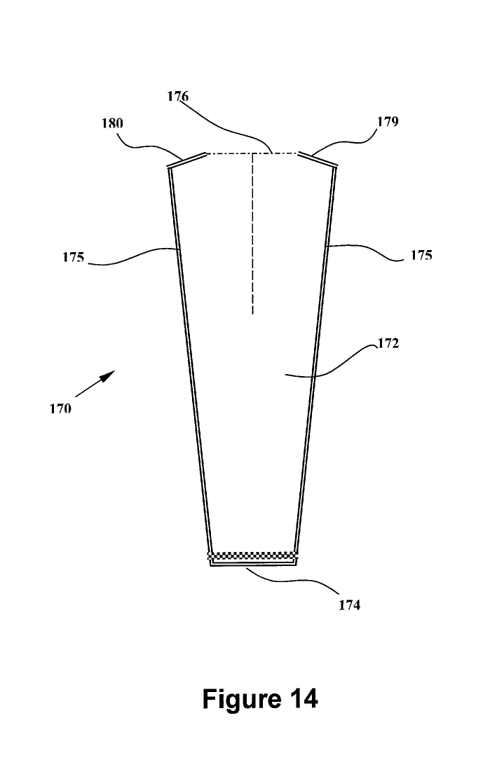

FIG. 14 shows another protective cover 170 that comprises a bag member 172 of a trapezium shape, with its first, closed end 174 slightly shorter than its second, open end 176. The protective cover 170 has two longitudinal sides 175, which are either with or without seam lines. Moreover, Such protective cover 170 comprises two transverse seals 179 and 180 at the extremities of the end section that includes the open end 176.

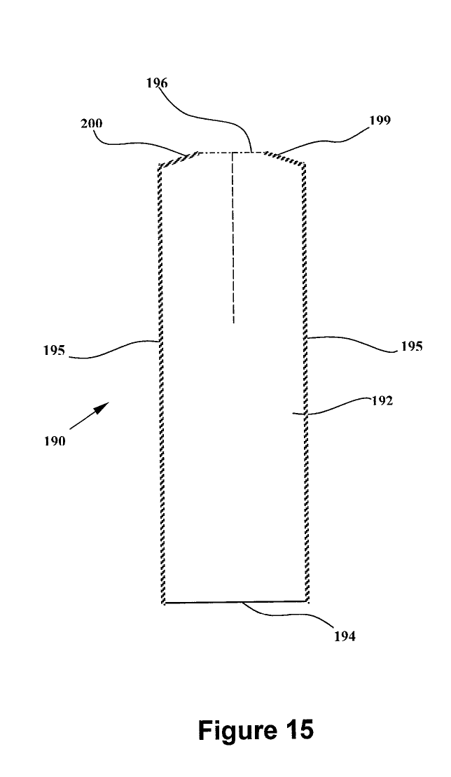

FIG. 15 shows yet another protective cover 190 according to one embodiment of the present invention, which comprises an elongate bag member 192. Such elongate bag member 192 are formed by folding an elongate strip of polymeric film along a transverse center line, and then sealing the two longitudinal sides 195 to form two seam lines, so that such elongate bag member 192 will have a close end 194 at the folding edge and an opposite, open end 196. It is preferred that such bag member are further partially sealed at the open end 196, to form two transverse seals 199 and 200 at the extremities of its open end portion, as shown by FIG. 15.

While the invention has been described herein with respect to various illustrative aspects, features and embodiments, it will be recognized that the invention is not thus limited, but that the present invention extends to and encompasses other features, modifications, and alternative embodiments, as will readily suggest themselves to those of ordinary skill in the art based on the disclosure and illustrative teachings herein. The claims that follow are therefore to be construed and interpreted as including all such features, modifications and alternative embodiments, within their spirit and scope.

What is claimed is:

1. A protective cover for a stethoscope or like article including a stethoscope head, an elongate connector body portion, and first and second ear tubes, said protective cover comprising:
an elongate bag member closed at a first end thereof and open at a second, opposite end thereof, defining an enclosed interior volume with an opening at said second, opposite end, wherein the stethoscope head and the elongate connector body portion of said stethoscope or like article are reposed in said interior volume of the bag member;
said bag member being longitudinally, medially severable along an end portion thereof including said second, opposite end, by application of tensional force at opposing margins of said end portion of the bag member, to yield respective free end segments for covering the first and second ear tubes of said stethoscope or like article.

2. The protective cover of claim 1, wherein the bag member is formed of a material that is acoustically transmissive and impermeable to viruses, bacteria, and fluids.

3. The protective cover of claim 1, wherein the bag member is formed of a thermoplastic polymeric material.

4. The protective cover of claim 1, wherein the bag member is closed at said first end thereof by a method selected from the group consisting of heat sealing, radio-frequency sealing, ultrasonic welding, and adhesive bonding.

5. The protective cover of claim 1, wherein the longitudinally, medially severable end portion of the bag member is scored for separation into said free end segments of the bag member.

6. The protective cover of claim 1, wherein the longitudinally, medially severable portion of the bag member comprises a central tear line for separation into said free segments of the bag member.

7. The protective cover of claim 1, wherein the bag member comprises two longitudinal sides that have seam lines.

8. The protective cover of claim 1, wherein the bag member are seamless along its two longitudinal sides.

9. The protective clover of claim 1, further comprising means for removably securing said bag member to said stethoscope or like article in a manner such that said stethoscope or like article is substantially covered by said bag member.

10. The protective cover of claim 1, further comprising means for associatively retaining the free end segments of the bag member in protective covering relationship to respective ear tubes of said stethoscope or like article.

11. The protective cover of claim 10 wherein said means for associatively retaining said free end segments in protective covering relationship to respective ear tubes of said stethoscope or like article, comprise transverse heat seals at extremities of the free end segments.

12. The protective cover of claim 10, wherein said means for associatively retaining the free end segments in protective covering relationship to respective ear tubes of said stethoscope or like article, comprise compressive retention elements.

13. The protective cover of claim 12, wherein said compressive retention elements comprise adhesive for adhesively securing said compressive retention elements to the bag member.

14. The protective cover of claim 10, wherein said means for associatively retaining said free end segments in protective covering relationship to respective ear tubes of said stethoscope or like article, comprise clip members.

15. The protective cover of claim 14, wherein said clip members comprise adhesive for adhesively securing said clip members to the bag member.

16. A protectively covered stethoscope assembly comprising:
a protective cover as in claim 1, and
a stethoscope having a stethoscope head, an elongate connector body portion, and first and second ear tubes,
wherein said stethoscope head and said elongate connector body portion are disposed in the interior volume of the bag member, and wherein said first and second ear tubes are disposed in respective free end segments of the bag member, so that the stethoscope is substantially shrouded by said bag member to prevent contamination by or transmission of biohazardous materials.

17. The stethoscope assembly of claim 16, wherein the longitudinally, medially severable end portion of the bag member is scored for separation into said free end segments of the bag member.

18. The stethoscope assembly of claim 16, wherein the longitudinally, medially severable portion of the bag member comprises a central tear line for separation into said free segments of the bag member.

19. The stethoscope assembly of claim 16, wherein the free end segments of the bag member are removably secured to respective ear tubes of the stethoscope.

20. A receptacle for dispensing multiple protective covers for stethoscopes or like articles, comprising:
a roll member that comprises a continuous elongate strip of multiple protective covers, each as in claim 1; wherein said protective covers are in lay-flat film form and arranged in series-connected relationship, with a transverse tear line between every two adjacent protective covers, said transverse tear line comprising a central slit; and
a box enclosure in which said roll member is disposed, said box enclosure having:
(c) a top hingedly attached thereto, said top comprising a tongue member capable of protruding exteriorly of said box enclosure for engaging the central slit of the transverse tear line between every two adjacent protective covers;
(d) a slot opening in a side wall of said box enclosure, through which a leading end portion of the continuous elongate strip of multiple protective covers is drawable, in such manner that the central slits of successive tear lines of said continuous elongate strip are in sequential register with the exteriorly protruding tongue member, whereby application of tensional force longitudinally along said continuous elongate strip effects severing of at least one protective cover from a rearwardly next-succeeding protective cover in said continuous elongate strip.

21. The receptacle of claim 20, wherein the box enclosure is formed of a material selected from the group consisting of paperboard, metals, metal alloys, plastics, and wood board.

22. The receptacle of claim 20, wherein the top of said box enclosure comprising a front flange, from which the tongue member is depending outwardly and against which said tongue member is foldable, in such manner that when the front flange is interiorly positioned against a front wall of the box enclosure, said tongue member protrudes vertically above the top of the box enclosure.

23. The receptacle of claim 20, wherein the slot opening of said box enclosure is in a rear wall of the box enclosure.

24. The receptacle of claim 20, wherein the slot opening of said box enclosure is in a front wall of the box enclosure.

25. The receptacle of claim 20, wherein said box enclosure further comprises a side compartment at one side thereof for containing a supply of compressive retention elements, said compressive retention elements associatively retaining said free end segments of the bag member in protective covering relationship to respective ear tubes of said stethoscope or like article.

26. The receptacle of claim 25, wherein said side compartment has an opening at the top of said box enclosure.

27. The receptacle of claim 25, wherein said side compartment has an opening at a sidewall of said box enclosure.

28. A method of protectively sheathing a stethoscope to prevent contamination by or transmission of biohazardous material in use of the stethoscope during a patient examination procedure, comprising protectively sheathing said stethoscope in a protective cover as in claim 1, prior to the patient examination procedure.
